(19) United States
(12) Patent Application Publication
(10) Pub. No.: US 2005/0266106 A1
(43) Pub. Date: Dec. 1, 2005

(54) Use of fermented wheat germ extract as anti-inflammatory agent

(76) Inventors: Mate Hidvegi, Budapest (HU); Akos Resetar, Budapest (HU)

Correspondence Address: BIRCH STEWART KOLASCH & BIRCH, PO BOX 747, FALLS CHURCH, VA, 22040-0747, US

(21) Appl. No.: 10/522,131

(22) PCT Filed: Aug. 8, 2003
(86) PCT No.: PCT/HU03/00065

§ 371 Date: Jan. 24, 2005

Related U.S. Application Data
(60) Provisional application No. 60/397,761, filed on Jul. 22, 2002.

(51) Int. Cl. A61K 35/78

(52) U.S. Cl. 424/750

(57) ABSTRACT

The present invention relates to a new therapeutic use of a fermented wheat germ extract under the trade name Avemar®, more specifically to use of Avemar® for the manufacture of a medicament useful as anti-inflammatory agent for preventing or treating or alleviating inflammatory conditions, particularly arthritis.

The present invention relates to a new therapeutic application of a fermented wheat germ extract under the trade name Avemar®, more specifically to the use of Avemar® for the manufacture of pharmaceutical compositions useful as anti-inflammatory agent for preventing or treating or alleviating inflammatory conditions, particularly arthritis.

The production method as well as the immunostimulant and anti-metastatic effects of the fermented wheat germ extract (hereinafter referred to as Avemar®) are described in WO 99/08694. This substance can be obtained by fermenting wheat germ with Saccharomyces cerevisiae in an aqueous medium and drying the filtered liquid ferment. The obtained substance is characterized by its 2.6-dimetoxi-p-benzoquinone content representing approximately 0.4 mg/g dry substance.

Surprisingly, it was found during our investigations that Avemar® can be applied for treating or preventing inflammatory diseases, particularly rheumatoid arthritis occuring in mammals including humans.

Arthritis is a general denomination for a number of arthritic diseases, such as rheumatoid arthritis, bacterial arthritis, reactive arthritis, etc. Rheumatoid arthritis includes a large group of non-bacterial states, the most important symptoms of which are the inflammation and deformation of joints. In most of the cases, classic rheumatoid arthritis affects a number of joints (polyarthritis), but it can also be limited to a single joint (monoarthritis). The attack of an arthritic cartilage is only one of the factors deforming numerous cartilages and bones and destroying articular function. This disease affects the articular sheath, ligaments and the bone tissue as well. In the majority of cases, the disease is characterized by varying courses, including aggravation and improvement periods accompanying the entire lifetime; however, the articular deformation and systemic disability continuously deteriorate. Only about 10% of the patients is spontaneously recovered.

The prevailing methods of treatment are directed to alleviate pain and reduce symptoms; there is no treatment at present leading to complete recovery from this disease. In most of the known treatment methods, anti-inflammatory agents are applied, such as steroids (prednisolon, dexamethasone), non-steroidal anti-inflammatory drugs (NSAIDs) and anti-rheumatic drugs affecting the disease (DMARDs). The NSAID group involves salicilates, ibuprofen, fenoprofen, naproxen, piroxicam, tolmetine, indomethacine and others, e.g. cyclooxygenase enzyme inhibitors. These chemotherapeutic drugs are characterized by little effectiveness and high toxicity.

The groups of DMARD or SAAR (anti-rheumatic drugs with prolonged effect) include D-penicillinamine, gold salts, chloroquine, asatioprine, methotrexate and cyclophosphamide. In view of their high toxicity, these drugs are usually selected by professionals only in the second place when the patient's responses are less favourable to NSAIDs. These agents are usually applied in combination with NSAIDs.

Recently, non-steroidal anti-inflammatory agents have also been developed for treating rheumatoid arthritis, including gamma-interferon and interleukin-6 antagonists, cyclosporine, PAF-antagonists, eicosapentaenoic acid (EPA), somatostatine analogues, peptide derivatives and immune modulators.

Hungarian patent No. 203044 describes a pharmaceutical preparation to ameliorate arthritis wherein the active agent is a herb extract.

Despite of the great number of medicines available at present, it is difficult or impossible to improve the status of many patients by medicinal treatment. Furthermore, there is no preventive method for rheumatoid arthritis.

Therefore there is an on-going demand for new types of anti-rheumatic drugs which are less toxic, produce less side effects and are suitable for eliminating, alleviating and preventing the symptoms of rheumatoid arthritis. New agents are needed to suppress or reduce inflammation, swelling and abnormal neovascularization, bone or articulation erosion which are well tolerated at the same time.

Therefore, the effect of Avemar® was examined in adjuvant arthritis (AA) possible to be triggered in rats which is the most frequently used experimental model of human rheumatoid arthritis (RA). It has been found that the development of adjuvant arthritis corresponds to human rheumatoid arthritis in several characteristics therefore it can be properly used for screening compounds. Mostly, this chronic inflammation model is used by pharmacologists to test the anti-inflammatory and immuno-suppressive effects of pharmacones.

The time and degree of AA development in rats depends on several factors, such as the triggering agent and its dose, the location of injection, the strain of experimental rat, etc. An acute inflammatory reaction (primary response) appears on the injected foot pad within 24 hours of administration and the volume of the paw gradually increases for 4 or 5 days. Depending on the strain of the used rat, the degree of inflammation becomes constant (plateau effect) between the 6^{th} and 11^{th} days; then the intensity of the reaction further increases. An inflammatory reaction is generated on the non-injected foot pad as well (a secondary or immune-mediated response) on the 10^{th} to 12^{th} day following the injection. The inflammatory reaction, that is, the increase in paw volume reaches the maximum on both the treated and the untreated foot pad between the 18^{th} and 21^{th} days.

### BRIEF DESCRIPTION OF DRAWINGS

FIG. 1 shows the effect of a 22-day p.o. treatment on AA (injected foot pad)

FIG. 2 shows the effect of a 22-day p.o. treatment on AA (non-injected foot pad)

FIG. 3 shows the effect of a 22-day p.o. treatment on AA on the 14^{th} day (injected foot pad)

FIG. 4 shows the effect of a 22-day p.o. treatment on AA on the 18^{th} day (injected foot pad)

FIG. 5 shows the effect of a 22-day p.o. treatment on AA on the 22^{nd} day (injected foot pad)

FIG. 6 shows the effect of a 22-day p.o. treatment on AA on the 14^{th} day (non-injected foot pad)

FIG. 7 shows the effect of a 22-day p.o. treatment on AA on the 18^{th} day (non-injected foot pad)

FIG. 8 shows the effect of a 22-day p.o. treatment on AA on the 22^{nd} day (non-injected foot pad)

FIG. 9 shows the effect of a 22-day p.o. treatment on the body weight of rats in function of time

FIG. 10 shows the effect of a 22-day p.o. treatment on the body weight of rats on the 22^{nd} day

FIG. 11 shows the effect of a 35-day p.o. treatment on AA (injected foot pad)

FIG. 12 shows the effect of a 35-day p.o. treatment on AA (non-injected foot pad)

FIG. 13 shows the effect of a 35-day p.o. treatment on AA on the 28^{th} day (injected foot pad)

FIG. 14 shows the effect of a 35-day p.o. treatment on AA on the 32^{nd} day (injected foot pad)

FIG. 15 shows the effect of a 35-day p.o. treatment on AA on the 35^{th} day (injected foot pad)

FIG. 16 shows the effect of a 35-day p.o. treatment on AA on the 28^{th} day (non-injected foot pad)

FIG. 17 shows the effect of a 35-day p.o. treatment on AA on the 32^{nd} day (non-injected foot pad)

FIG. 18 shows the effect of a 35-day p.o. treatment on AA on the 35^{th} day (non-injected foot pad)

FIG. 19 shows the effect of a 35-day p.o. treatment on the body weight of rats in function of time

FIG. 20 shows the effect of a 35-day p.o. treatment on the body weight of rats on the 35^{th} day

FIG. 21 shows severe chronic inflammatory infiltration in the synovium and adjacent tissues of untreated rats presenting AA

FIG. 22 shows severe infiltration containing giant cells in the synovium of untreated rats presenting AA

FIG. 23 shows micro-abscesses within an inflammatory infiltration in the periarticular tissue of untreated rats presenting AA

FIG. 24 shows CD4 positive lymphocytes in the inflammatory infiltration found in the synovium of untreated rats presenting AA

FIG. 25 shows the lack of inflammatory infiltration in the synovial and perisynovial tissues of rats previously presenting AA and treated by Avemar.

Effect of Avemar® on Adjuvant Arthritis in Rats

In our experiments, adjuvant arthritis was triggered in female Wistar rats by injecting 0.1 ml 0.5% killed Mycobacterium butyricum (Difco) suspended and homogenized in liquid paraffin under the skin of the sole of the right hind paw of the animals. The average initial body weight of the experimental animals was 138±5 g in the group treated for 22 days and 118±5 g in the group treated for 35 days. The body weight of the animals was measured by plethysmography during the treatment by Avemar®, together with the volume of the injected right leg and the non-injected left leg (to follow the changes of the primary and secondary reactions) on days 0, 1, 4, 7, 12, 14, 18 and 22 in the 22-day experiment and on days 0, 3, 7, 11, 15, 18, 21, 25, 28, 32 and 35 in the 35-day experiment. The inflammatory reaction was triggered on day 1 (22-day test) and on day 14 (35-day test), respectively, after starting treatment by Avemar®. The following experimental groups and methods were applied in both experiments: 1. Control 2×1.0 ml/150 g (distilled water); 2. Avemar® 2×2.5 g/kg/day; 3. Avemar® 2×1.0 g/kg/day; 4. Avemar® 2×0.25 g/kg/day; 5. Avemar® 2×0.05 g/kg/day; 6. Indomethacin 2×0.5 mg/kg/day; 7. Dexamethasone 2×0.05 mg/kg/day. Each experimental group consisted of 10-16 rats.

Suspensions with Avemar® (manufactured by Biromedicina Pcl., Budapest, Hungary) and dexamethasone solutions were always prepared and/or diluted immediately before the administration. Indomethacin (manufactured by Chinoin Pharmaceutical and Chemical Works, Budapest, Hungary) applied as a positive control was suspended in 0.5% carboxymethylcellulose and administered. The various doses of Avemar® as well as indomethacin and dexamethasone (manufactured by Organon) were administered by gastric tube in 1.0 ml/150 g body weight twice a gay, i.e. the first half of the daily dose between 8.00 and 10.00 a.m. and the other half between 4.00 and 6.00 p.m. The control group received 1 ml/150 g distilled water.

Single-way analysis of variance (ANOVA) was performed for the statistical evaluation of the results.

Results

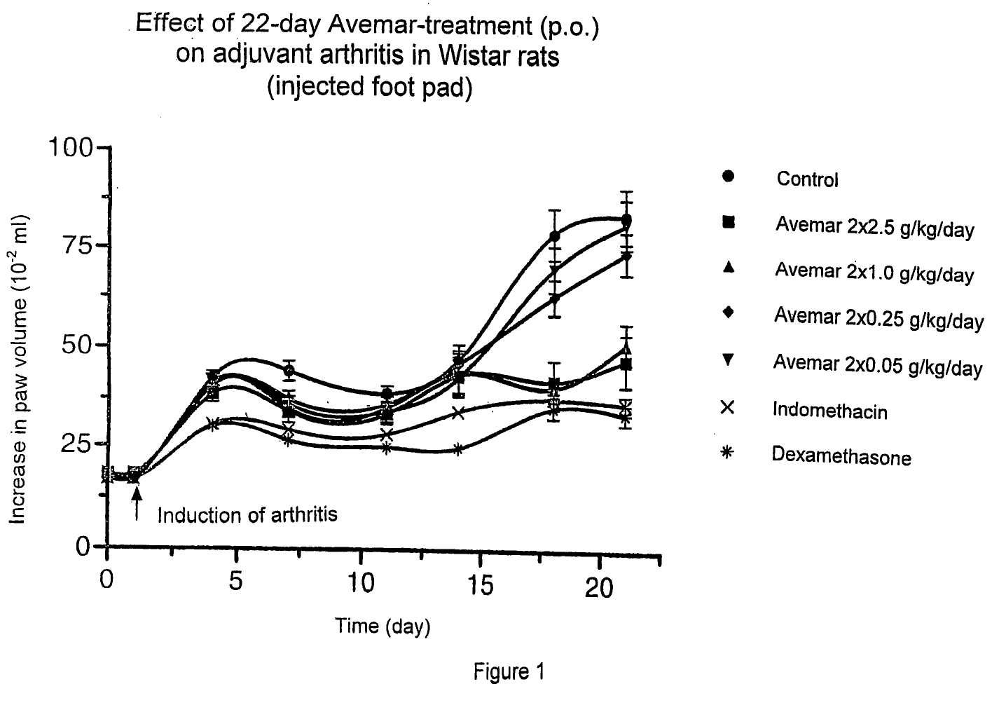

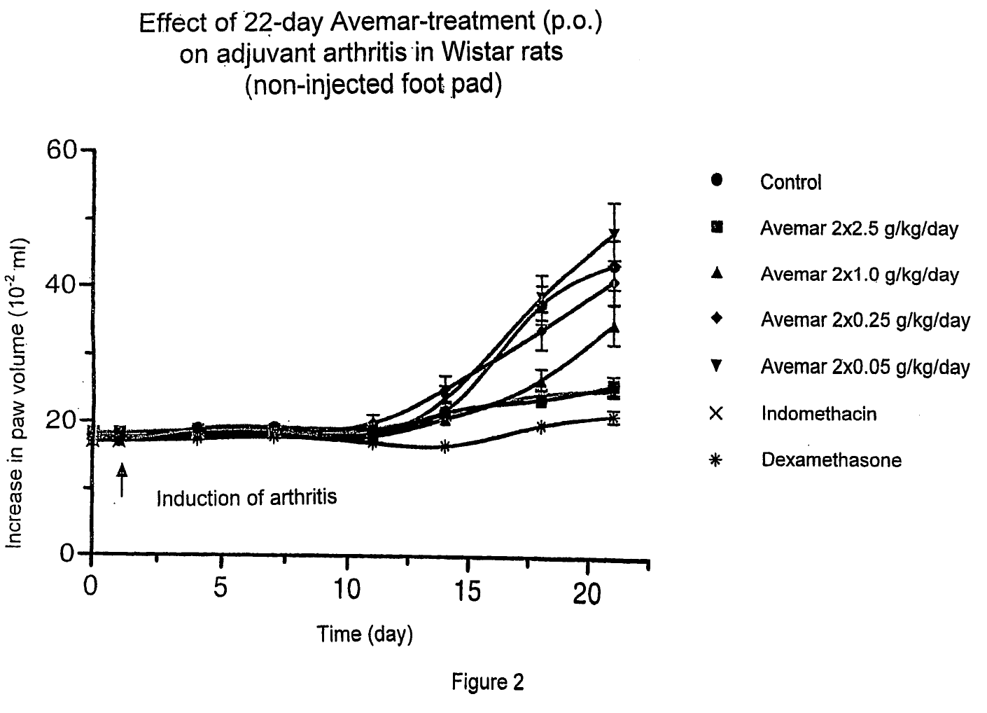

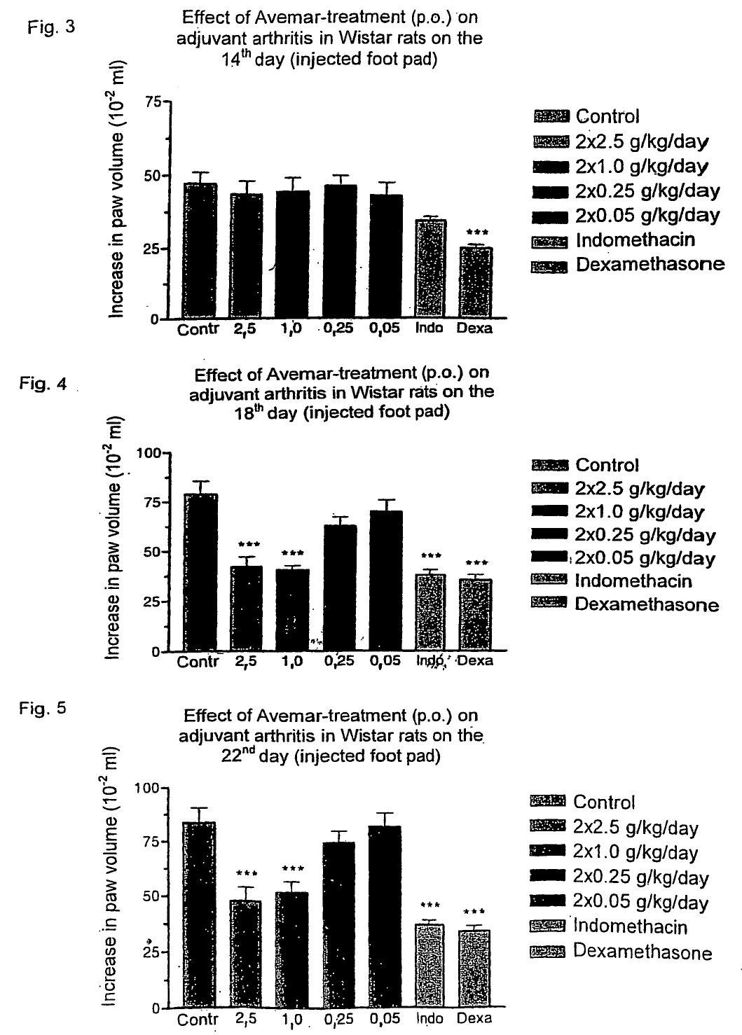

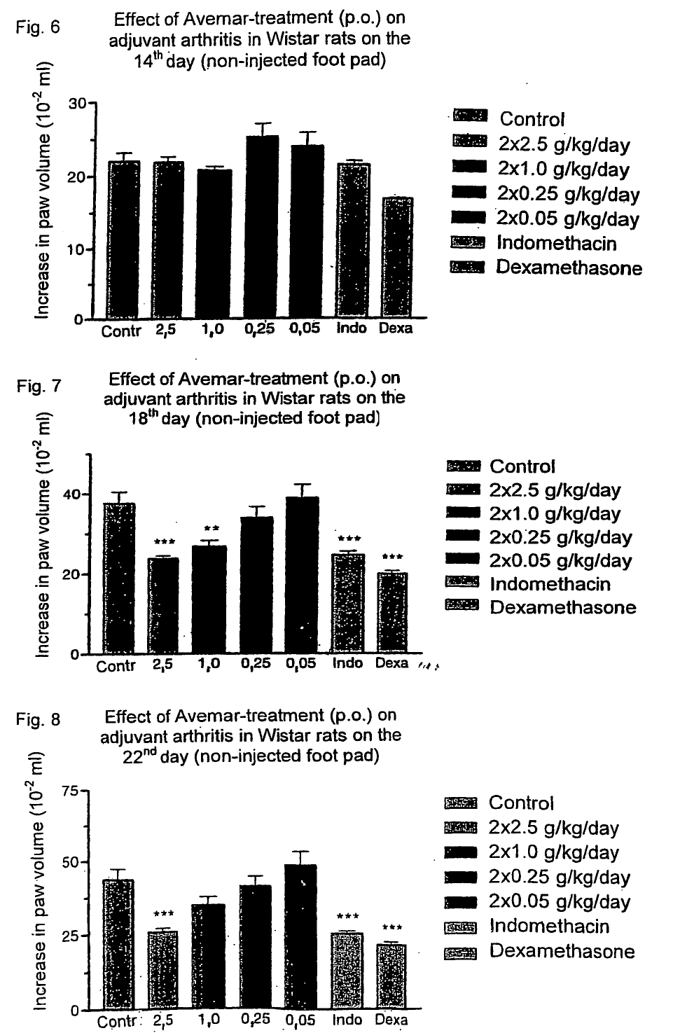

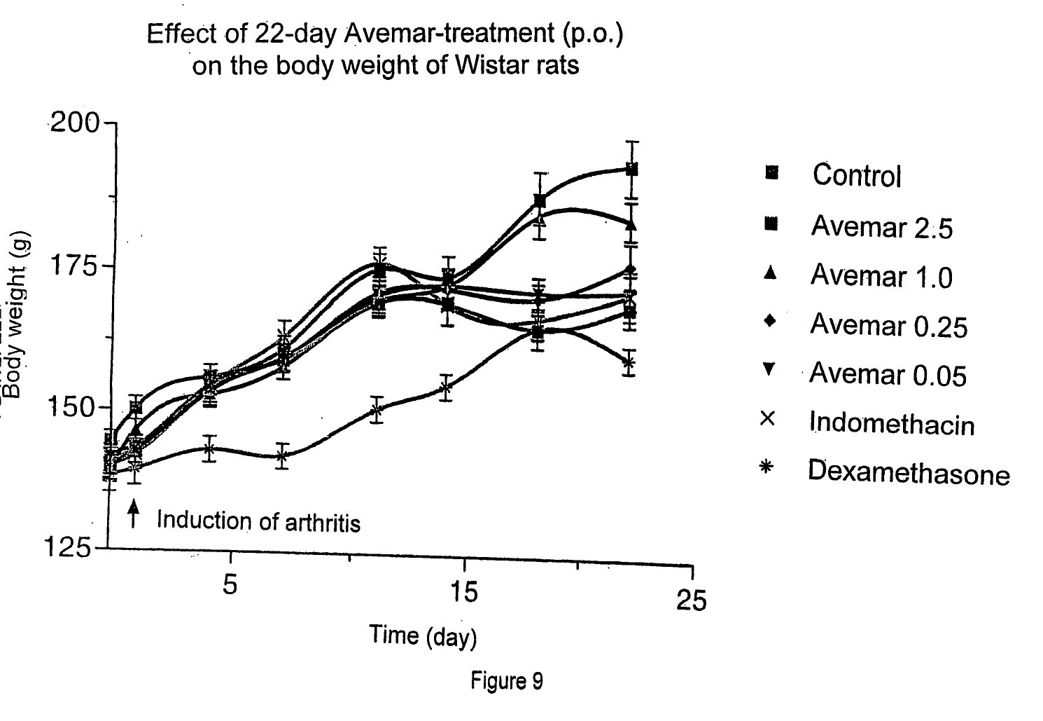

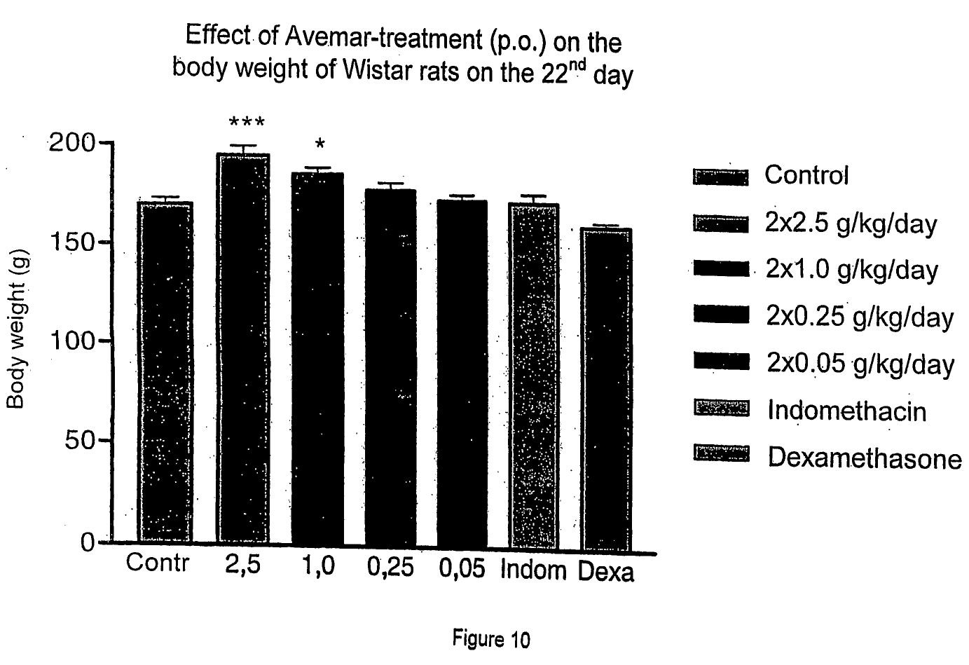

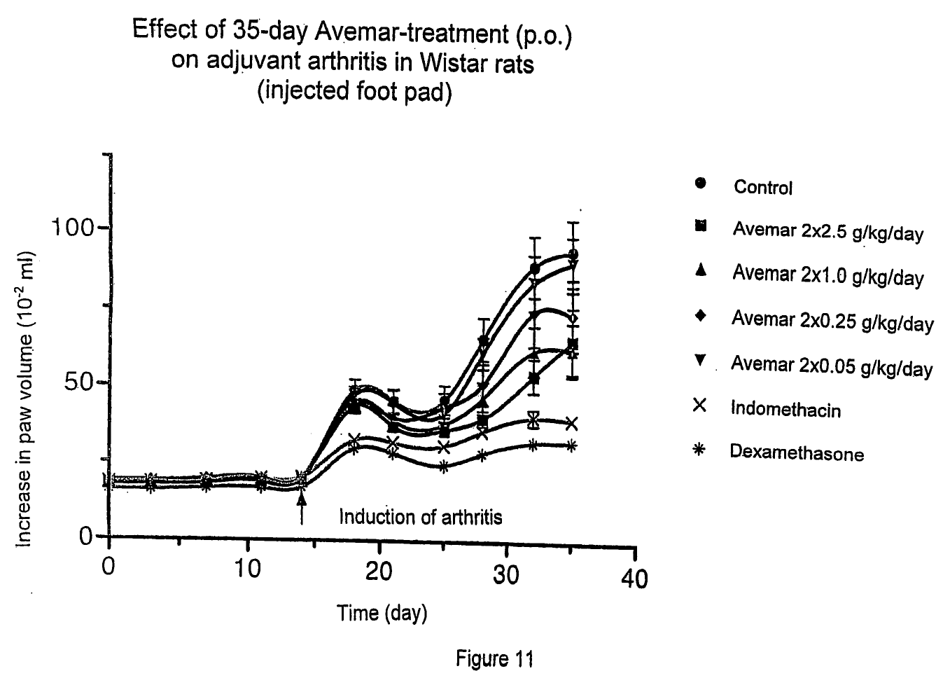

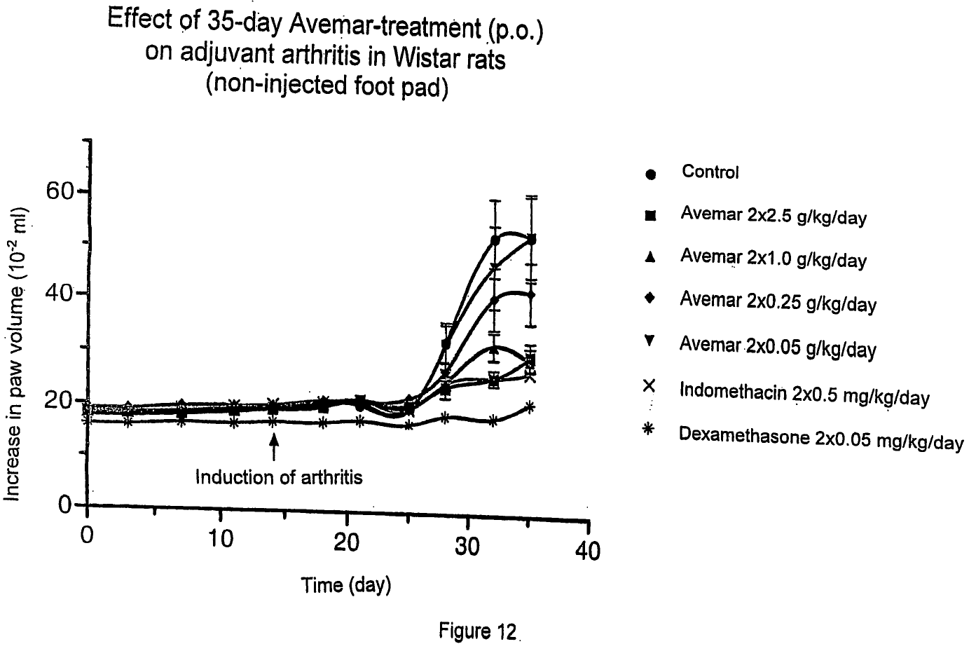

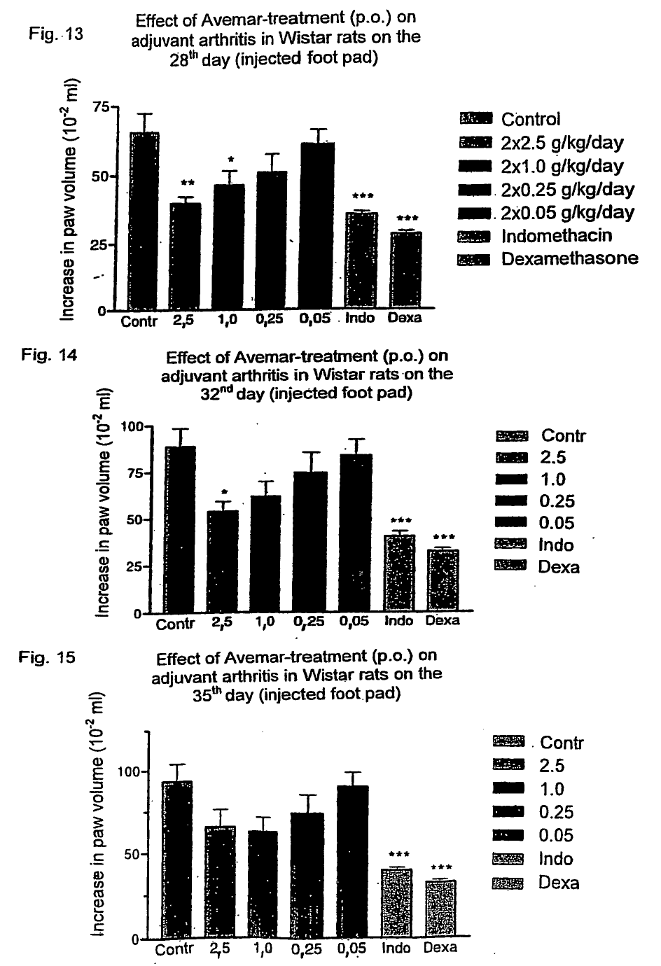

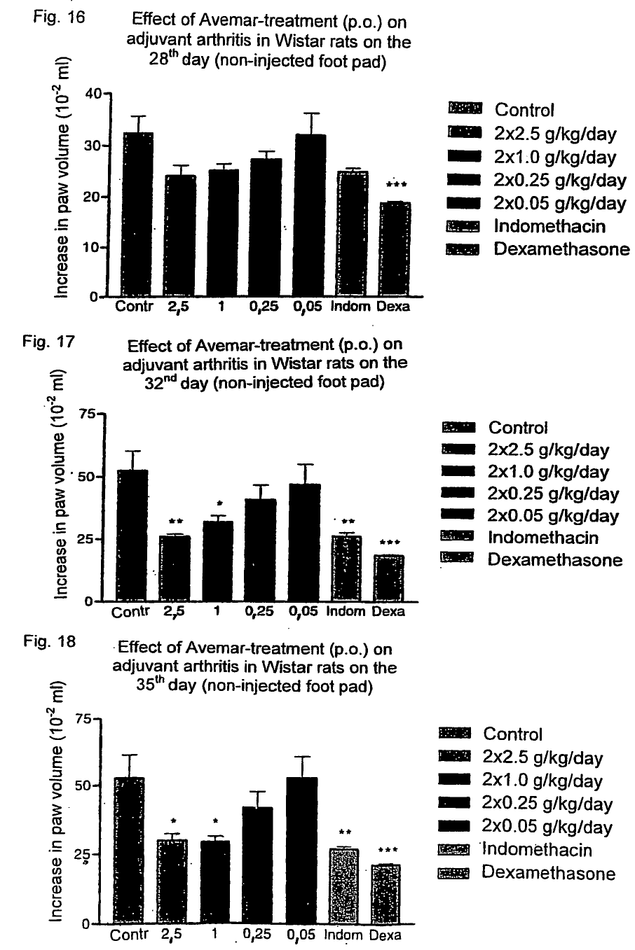

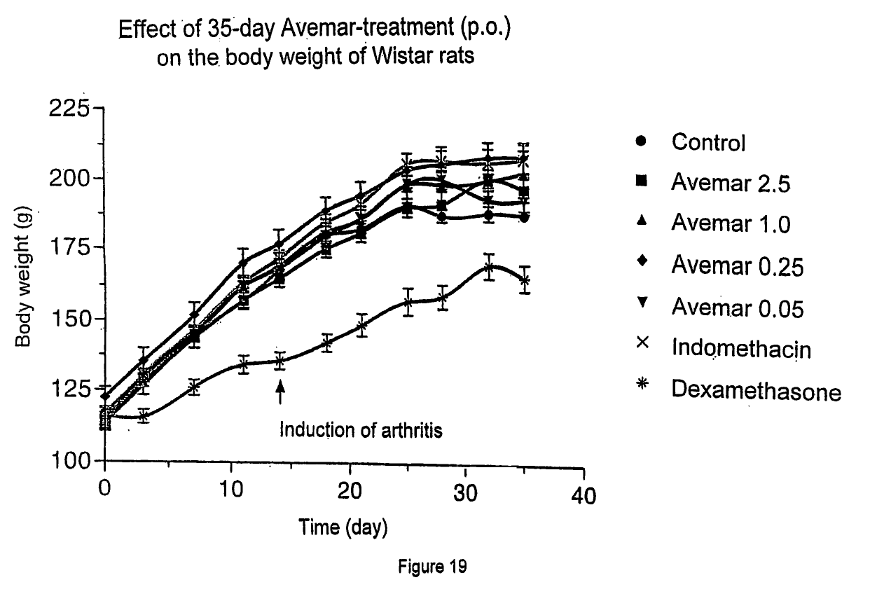

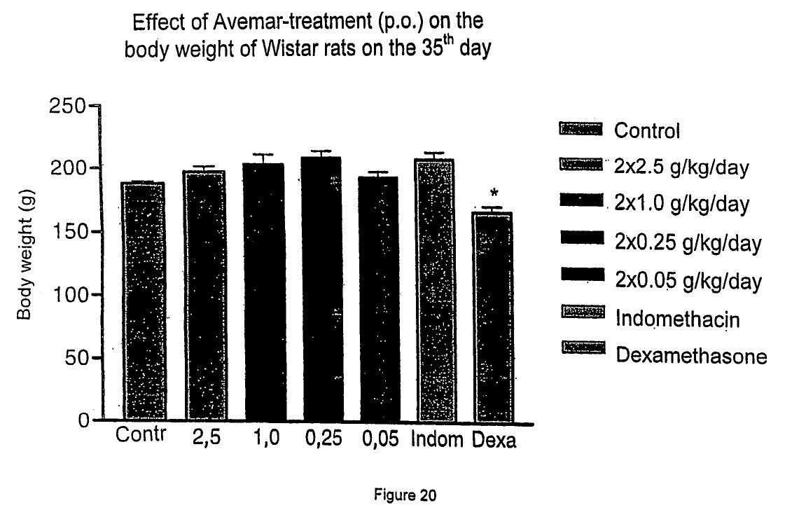

Results of the 22-day treatment are shown in FIGS. 1 to 10 and the results of the 35-day treatment in FIGS. 11 to 20. The figures show group average values with standard deviation (±SEM) (for 22-day experiments n=14-15 and for 35-day experiments n 10-12). Significance levels compared to the control group were indicated by * over the bar charts (*=p<0.05; **=p<0.01; ***=p<0.001).

The results show that Avemar® can depending on the dose significantly inhibit the development of both the primary and the secondary inflammatory reactions in rats which supports its anti-inflammatory effect. Similarly to indomethacin and dexamethasone, Avemar® minimized adjuvant arthritis in a dose-dependent manner in the treated rats. (Although its effectiveness does not achieve that of indomethacin and dexamethasone applied as positive controls, it is still capable of significantly inhibiting adjuvant arthritis.) During the 14-day pretreatment, it does not influence leg volume, which means that it does not cause a generalized inflammatory response. Depending on the duration of pretreatment, it inhibits the development of arthritis. Pretreatment by Avemar® cannot or can only slightly increase the body weight of rats with arthritis during a 22-day or 35-day treatment.

Histological Studies

The affected joints of the right hind foot pad together with the epiphyses of the bones and the surrounding fibrous and muscular tissues were fixed in buffered neutral 4% formalin. Decalcification was performed by using EDTA and the samples were embedded into paraffin. 8 μm thin longitudinal sections were cut and the sections were stained with hemotoxiline (H) and eosine (E). In the selected positive control and in the treated cases an immunoperoxidase reaction was performed to show CD4 and CD8 positive T-lymphocytes. The used antibody was the product of Santa Cruz (Santa Cruz, Calif., USA), applied in 1:100 dilution.

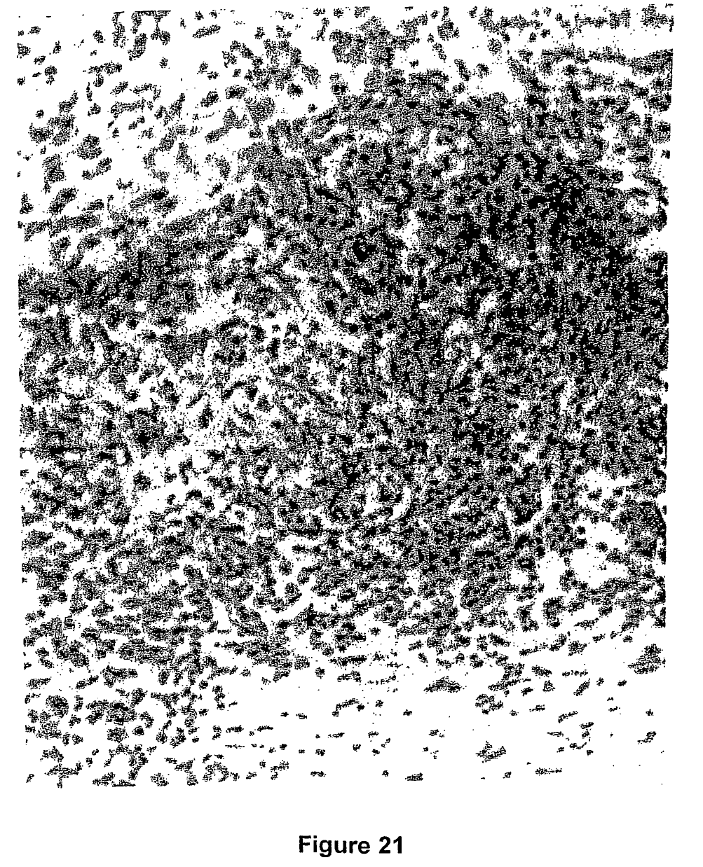

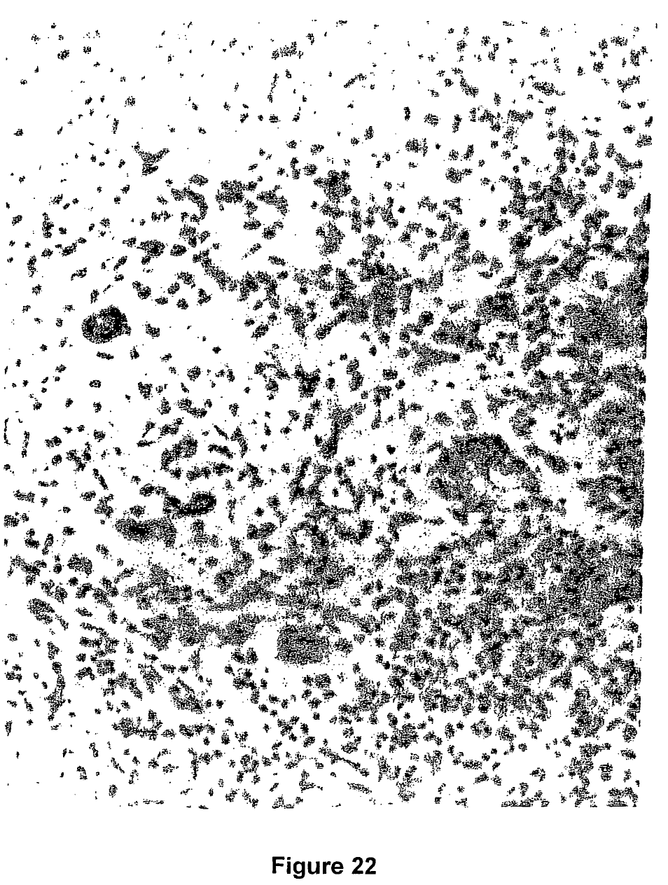

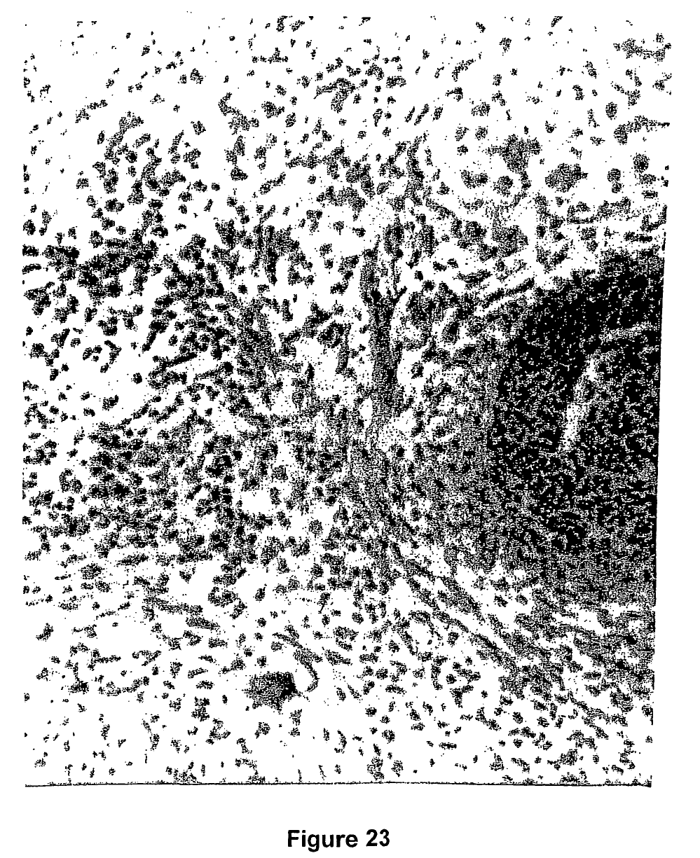

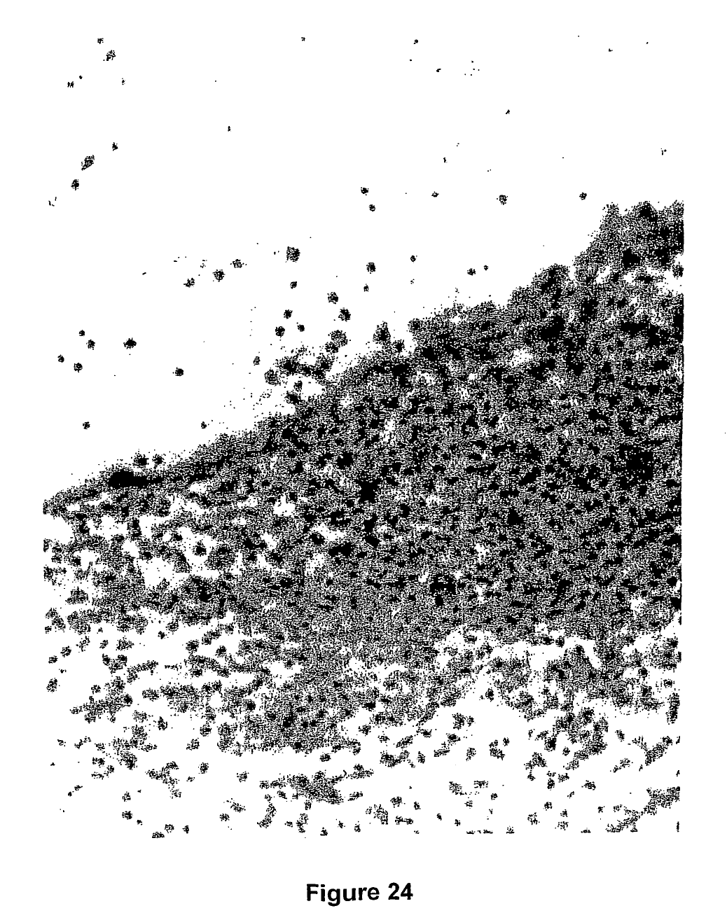

The histological examination of the joints of untreated control rats suffering from AA revealed severe inflammatory changes of the synovium and the surrounding (perisynovial) tissues (FIG. 21, stained by H and E, magnified 300 times). The cellular infiltration consisted of lymphocytes, plasma cells, histiocytes, multi-nucleated giant cells and fibroplasts (FIG. 22, stained by H and E, magnified 300 times). Within the inflammatory infiltration micro-abscesses formed by neutrophil granulocytes were also observed (FIG. 23, stained by H and E, magnified 300 times). The majority of lymphocytes proved to be CD4 positive in the inflammatory infiltrates of AA rats (FIG. 24, CD4 immunoperoxidase).

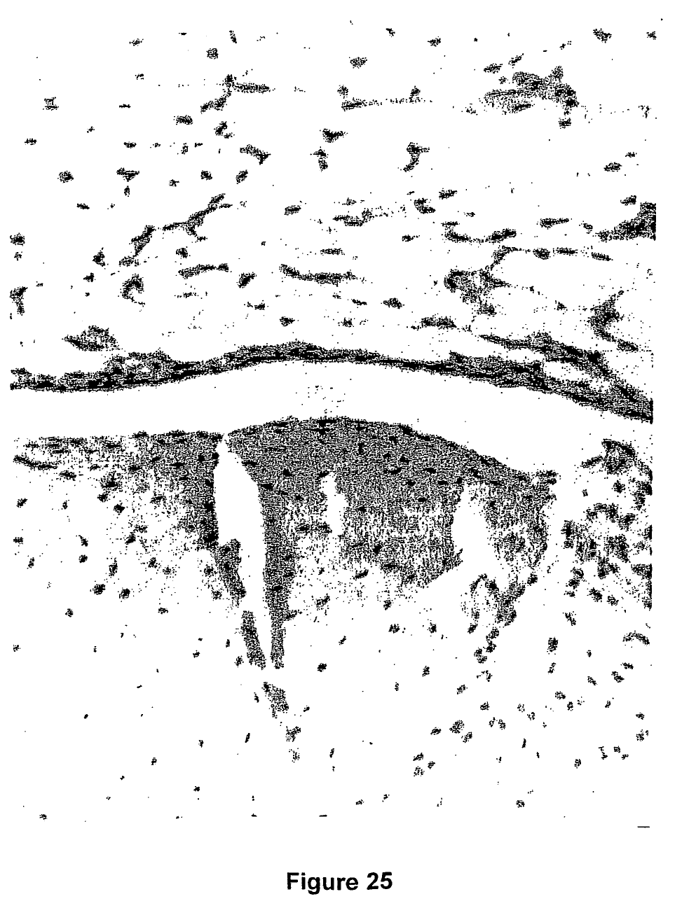

The joints of the rats treated by Avemar® 2×1.0 or 2×2.5 g/kg/day showed no or minimal inflammatory infiltration. The infiltration of CD4 positive lymphocytes in synovial and perisynovial cells almost totally disappeared and fibrosis was minimized as a result of treatment by Avemar® (FIG. 25, stained by H and E, magnified 300 times). Similar results were found in animals treated by indomethacin and dexamethasone used as positive controls. The semi-quantitative estimation of the degree of inflammatory infiltrates in the different groups is shown in Table 1. There was no significant difference between the groups receiving 24-hour and 14-day pretreatment, respectively.

TABLE 1
Semi-quantitative histological qualification of inflammatory
infiltrates in AA rats untreated and treated by Avemar,
indomethacin or dexamethasone

| | Histological grade (average 1 to 3) | |
|---|---|---|
| | 24-hour | 14-day |
| Treatment | pretreatment | pretreatment |
| Control | 2.8 | 2.2 |
| 2 × 2.5 g/kg/day Avemar ® | 1.6 | 1.4 |
| 2 × 1.0 g/kg/day Avemar ® | 1.0 | 1.2 |
| 2 × 0.25 g/kg/day Avemar ® | 2.4 | 2.6 |
| 2 × 0.05 g/kg/day Avemar ® | 2.6 | 2.6 |
| 2 × 0.5 mg/kg/day | 1.0 | 1.2 |
| Indomethacin | | |
| 2 × 0.05 mg/kg/day | 1.4 | 1.2 |
| Dexamethasone | | |

Consequently, histological studies unequivocally support the anti-inflammatory effect of Avemar®.

On the basis of these results it can be assumed that the development of rheumatoid arthritis can be inhibited by an appropiate dosage of Avemar®.

Acute and sub-acute toxicity tests performed under GLP (Good Laboratory Practice) conditions showed that unlike the steroidal and non-steroidal anti-inflammatory compounds Avemar® did not exert any toxic effect, including erosive gastritis and acute gastric ulcer (Report. Acute oral toxicity study of Avemar® in mice. Code: 9901. Univ. Vet. Sci., Dept. Pharmacol. Toxicol., Budapest, 1999; Acute oral toxicity study of Avemar® in rats. Code: 9902. Univ. Vet. Sci., Dept. Pharmacol. Toxicol., Budapest, 1999; Subacute oral toxicity study of Avemar®. Code: 0001. Univ. Vet. Sci., Dept. Pharmacol. Toxicol., Budapest, 2000). Furthermore, the preparation was not genotoxic in micronucleus tests of rat bone marrow.

Based on the experimental results, it was suggested that Avemar® may be a suitable therapeutic tool in the treatment of rheumatoid arthritis in humans. Other immunopathological diseases may also be considered in this respect.

Effect of Avemar® on Human Rheumatoid Arthritis

Therapy-resistant patients proved to be suffering from rheumatoid arthritis were treated by Avemar® at Department IV of Rheumatology of the National Institute for Rheumatology and Physiotherapy (Budapest, Hungary). This open clinical test with self-control was aimed to assess the effectiveness, tolerability and side effects of Avemar®. The following is an account of the experiment results gained during the one-year treatment of 15 patients.

A. Selection of Patients

15 outpatients classified into Steinbrocker anatomic stages II to III proved to suffer from RA based by their ACR classification criteria participated in the study. Their RA had stagnated or aggravated under a therapy set for 3 months before starting treatment by Avemar®. The patients consented to participe in the study.

B. Criteria for Exclusion
age below 18 and over 80;
serious diseases of the liver, heart, kidney, and haematopoietic organs;
active gastric ulcer;
psychiatric diseases, mental backwardness;
lack of cooperation;
gravidity or lactation.
   In the event that the preparation is ineffective, that is, if no expected improvement comes about after 3 months, administration thereof will be suspended.

C. Course of the Study

Besides the original medicinal treatment (base therapy, steroid, and NSAID), the 15 RA patients were administered a daily dose of 2×9 g water soluble granulated Avemar® (9 g in the morning and 9 g in the evening). The patients were checked at the time of starting treatment and every month; their statistical evaluation was performed in months 6 and 12, respectively.

D. Test Parameters:

The clinical parameters of the study were as follows:
Ritchie index;
HAQ (Health Assessement Questionnaire);
duration of morning anchylosis in hours;
sedimentation;
CRP;
haematocryte value.

During the study the change of the administered steroid dose was also recorded. The average age of the 15 female RA patients participating in the study was 54.5 years (44-68 years) with an average disease duration of 8 years (3-25 years); all but one were seropositive. At the beginning of the study, 10 of the patiens received baseline therapy: 1 patient was treateed with sulphasalazine (Salazopyrin); 5 patients with methotrexate (Methotrexate, Lachema); 3 patients with cyclosporine (Sandimmun, Neoral); and 1 patient with chloroquine (Delagil). 5 patients did not receive base therapy: the base therapy drugs applied earlier had not proved to be effective and/or produced side effects. At the beginning of the study 11 patients were administered steroids and the highest oral steroid dose was 7.5-10 mg prednisolone or an equivalent dose of methylprednisolone or dexamethasone. Patient characteristics are shown in Table 2.

TABLE 2
Data of patients before start of Avemar ® treatment

| | | Duration | Stein- | | | | | | | Daily | | | |
|---|---|---|---|---|---|---|---|---|---|---|---|---|---|
| | | of | brocker | | | | | Morning | | dose of | | | |
| Patient | Age | disease | classi- | ESR | | HCT | Ritchie | anchylosis | | steroid | | Baseline therapy | |
| No. | (year) | (year) | fication | (mm/hr) | CRP | L/L | index | (hrs) | HAQ | (mg) | | dose/day | |
| 1 | 53 | 9 | 3 | 54 | 8 | 0.36 | 36 | 0 | 1.7 | 6 | M | 12.5 | mg/week MTX |
| 2 | 68 | 7 | 3 | 70 | 17 | 0.33 | 40 | 5 | 1.6 | 2 | M | — | |
| 3 | 62 | 12 | 3 | 40 | 5 | 0.35 | 28 | 3 | 1.0 | 0.8 | D | — | |
| 4 | 54 | 5 | 3 | 76 | 30 | 0.38 | 34 | 2 | 2.1 | — | | 2 | g S |
| 5 | 50 | 25 | 2 | 36 | 10 | 0.35 | 24 | 2.5 | 2.1 | — | | — | |
| 6 | 44 | 5 | 3 | 46 | 10 | 0.31 | 20 | 1 | 1.2 | 10 | P | 7.5 | mg/week MTX |
| 7 | 68 | 3 | 3 | 24 | 6 | 0.40 | 4 | 1.5 | 1.1 | — | | — | |
| 8 | 50 | 3 | 2 | 20 | 5 | 0.30 | 10 | 1 | 0.8 | 4 | T | 7.5 | mg/week MTX |
| 9 | 58 | 5 | 2 | 90 | 18 | 0.31 | 20 | 2.5 | 1.1 | 10 | P | — | |
| 10 | 44 | 4 | 2 | 36 | 11 | 0.38 | 11 | 1.5 | 0.6 | 5 | P | 175 | mg C |
| 11 | 50 | 6 | 3 | 18 | 6 | 0.33 | 10 | 1 | 0.8 | 4 | M | 250 | mg Ch |
| 12 | 60 | 5 | 3 | 3 | 106 | 0.37 | 16 | 5 | 2.6 | 6 | M | 200 | mg C |
| 13 | 48 | 5 | 2 | 84 | 182 | 0.37 | 32 | 6 | 1.8 | 6 | M | 225 | mg C |
| 14 | 50 | 25 | 3 | 40 | 16 | 0.36 | 24 | 0 | 1.6 | 4 | M | 12.5 | mg/week MTX |

| | | | | | | | | | | | | |
|---|---|---|---|---|---|---|---|---|---|---|---|---|
| 15 | 58 | 4 | 2 | 56 | 14 | 0.38 | 12 | 1.5 | 0.8 | — | 7.5 | mg/week MTX |

Steroids
Dexamethasone: D
Methylprednisolone: M
Triamcinolone: T
Prednisolone: P
Baseline therapy agents
Chloroquine: Ch
Methotrexate: MTX
Cyclosporine: C
Sulfasalazine: S
ESR: erythrocyte sedimentation rate
HAQ: Health Assessment Questionnaire

Results

Statistical calculations were performed using the Wilcoxon test. Results are shown in Tables 3 to 9.

TABLE 3
Change of Ritchie index compared to
the initial value in 6 and 12 months

| | | |
|---|---|---|
| Time | Z value | Significance |

| | | | |
|---|---|---|---|
| 0-6 | months | 2.574 | p < 0.010 |
| 0-12 | months | 2.953 | p < 0.003 |
| 6-12 | months | 0.534 | p < 0.594 N.S. |

TABLE 4
Change of HAQ value compared to the
initial value in 6 and 12 months

| | | |
|---|---|---|
| Time | Z value | Significance |

| | | | |
|---|---|---|---|
| 0-6 | months | 3.020 | p < 0.003 |
| 0-12 | months | 2.448 | p < 0.014 |
| 6-12 | months | 1.433 | p < 0.152 N.S. |

TABLE 5
Change of duration of morning anchylosis compared
to the initial value in 6 and 12 months

| Duration | | | | | | |
|---|---|---|---|---|---|---|
| of | | | | | Intensi- | Signifi- |
| treatment | | Stopped | Improved | Unchanged | fied | cance p |
| 0-6 | mths | 2 | 9 | 2 | 0 | 0.009 |
| 0-12 | mths | 2 | 9 | 1 | 1 | 0.002 |

TABLE 6
Change of sedimentation compared to
the initial value in 6 and 12 months

| | | |
|---|---|---|
| Time | Z value | Significance |

| | | | |
|---|---|---|---|
| 0-6 | mths | 2.131 | p < 0.033 |
| 0-12 | mths | 1.250 | p < 0.211 N.S. |
| 6-12 | mths | 0.559 | p < 0.576 N.S. |

TABLE 7
Change of CRP level

| | | |
|---|---|---|
| Time | Z value | Significance |

| | | | |
|---|---|---|---|
| 0-6 | mths | 1.318 | p < 0.187 N.S. |
| 0-12 | mths | 0.426 | p < 0.670 N.S. |
| 6-12 | mths | 0.565 | p < 0.572 N.S. |

Comparisons were made with T-tests of a single sample.

TABLE 8
Change of haematocryte value

| | | |
|---|---|---|
| Time | Z value | Significance |

| | | | |
|---|---|---|---|
| 0-6 | mths | 1.494 | p < 0.157 N.S. |
| 0-12 | mths | 3.011 | p < 0.009 |
| 6-12 | mths | 0.722 | p < 0.482 N.S. |

Comparisons were made with T-tests of a single sample.

TABLE 9
Change of steroid dose in 6 and 12 months

| Duration of | | Unchanged | | Increased | Signifi- |
|---|---|---|---|---|---|
| treatment | | dose | Reduced dose | dose | cance, p |
| 0-6 | mths | 5 | 6 | 0 | 0.031 |
| 0-12 | mths | 5 | 5 | 1 | 0.116 N.S. |

4 patients did not receive steroid.

5 out of the 11 patients taking steroid had their initial doses of medication unchanged; 2 patients managed to reduce the original quantity to half, and 2 patients from 7.5 mg to 5 mg. The dose of base therapy drugs changed at 4 patients in the 12-month test period: at one of them, MTX increased from 12.5 mg to 15 mg, and at the other three the dose of cyclosporine was reduced from 175 to 125 mg, from 225 to 100 mg, from 200 to 100 mg. The dose was unchanged at 5 patients.

No significant change was detected in haemoglobin, liver, and kidney function laboratory parameters.

The resulting data show that the treatment of therapy resistant RA patients by Avemar® yielded surprisingly favourable results in the first six months. As regards the patients' clinical parameters, significant improvement was brought about without exception; moreover, it was also possible to reduce the steroid dose of patients. By the end of the second half of the treatment, the significant improvement of clinical parameters still improved compared to the initial status.

Based on the above, the object of the present invention is the use of fermented wheat germ extract (Avemar®) for preparing pharmaceutical compositions for treating or preventing or alleviating inflammatory conditions.

Preferably, Avemar® can be applied to prepare pharmaceutical compositions useful for treating or preventing or alleviating arthritis, more preferably rheumatoid arthritis.

A further object of the present invention is a process for preparing pharmaceutical compositions containing fermented wheat germ extract as an active ingredient comprising manufacturing said active ingredient with commonly used pharmaceutically additives to a pharmaceutical composition useful for treating or preventing or alleviating inflammatory diseases.

Furthermore, it has been found that Avemar® can be applied prferably together with other non-steroidai (NSAID) type anti-inflammatory agents, such as diclophenac, ibuprophen, piroxicam, tolmetin, etc. As a result of co-administration, the dose of NSAID type drugs can be considerably reduced, which is a great advantage regarding the toxicity of these drugs. For example, the co-administration with diclophenac allows reducing by 50% the quantity of both agents and attaining similar effects of improvement the same time.

On the basis of the above, a further object of the present invention is the use of a fermented wheat germ extract (Avemar®) and another active ingredient, especially an anti-inflammatory agent for producing a medicament for treating or preventing or alleviating arthritis. According to the invention a non-steroidal anti-inflammatory agent is preferably used as another anti-inflammatory agent.

Furthermore, the present invention also relates to a combined pharmaceutical composition containing an effective amount of fermented wheat germ extract (Avemar®) in combination with another active ingredient, especially an anti-inflammatory agent and a pharmaceutically acceptable carrier.

Preferable anti-inflammatory pharmaceutical compositions of the invention contain an effective dose of fermented wheat germ extract (Avemar®) and diclofenac.

The active ingredient used in the present invention can be formulated in several oral and parenteral dosage forms and administered to treat and prevent rheumatoid arthritis. In general, the active ingredient is present in about 5% to 95% by weight in the composition.

The pharmaceutically acceptable excipients used for producing pharmaceutical compositions can be in solid or liquid phase. Examples of solid pharmaceutical compositions include powders, tablets, pills, capsules, cachets, rhomboid medicinal formulas, suppositories and dispersable granules. Solid compositions can include several additives such as thinners, flavors, soluble agents, lubricants, suspending agents, binders, preservatives, tablet desintegrators or encapsulating substances.

As regards powders, the excipient is a finely powdered solid substance which constitutes a mixture with the finely dispersed active ingredient.

As regards tablets, a carrier possessing the required binding characteristics is mixed in proper proportion with the active ingredient and pressed to the required shape and size.

Preferably, powders and tablets contain the agent in 5% to 70%. Examples of suitable excipients include magnesium carbonate, magnesium stearate, talcum, sugar, lactose, pectin, dextrin, cyclodextrin, maltodextrin, starch, gelatine, tragacanta, methylcellulose, sodium carboxymethylcellulose, waxes of low melting point, cocoa butter, etc. The production involves the formulation of the active ingredient with the encapsulating substance as excipient, thereby a capsule is obtained in which the active ingredient with or without other carriers is surrounded by the excipient, which the latter being thus linked to the active ingredient. Cachets and rhomboid drug formulas are produced similarly. Tablets, powders, capsules, pills, cachets and rhomboid drug formulas can be applied for oral administration.

In order to produce suppositories, waxes with low melting point, e.g. a mix of fatty acid glycerides or cocoa butter are first melted and the active ingredient is homogeneously dispersed therein by mixing. Then the melted homogeneous mix is poured into suppository moulds of appropriate size, left to cool down and solidify.

Liquid pharmaceutical preparations include solutions, suspensions, emulsions, syrups, and elixirs, such as aqueous or aqueous propylene glycol solutions. For parenteral injections, liquid pharmaceutical compositions can be formulated in an aqueous polyethylene glycol solution.

Solutions suitable for oral administration can be produced by dissolving the active ingredient in water and adding appropriate colorants, flavors, stabilizers and coagulants.

Suspensions suitable for oral administration can be produced by dispersing the finely ground active ingredient in water together with a viscous substance such as natural or synthetic rubbers, resin, methylcellulose, sodium carboxymethylcellulose and other well-known suspending agents.

Solid pharmaceutical compositions also include those intended to be converted into liquid preparations shortly before use for oral administration. Examples for such liquid pharmaceutical formulations include solutions, suspensions and emulsions. Besides the active ingredient, these pharmaceutical preparations may contain colorants, flavors, stabilizers, buffers, artificial and natural sweeteners, dispersing agents, coagulants, soluble agents and similar substances.

Sterile compositions for parenteral administration can be preferably aqueous or non-aqueous solutions, suspensions or emulsions. The following can be applied as solvents: water, propylene glycol, some sort of polyethylene glycol, vegetable oils, such as olive oil, injectable organic esters, such as ethyl oleate. These compositions may also contain other auxiliaries, particularly lubricants, isotonising, emulgeating, dispersing and stabilizing agents. Sterilization can be performed in several ways, including aseptic filtration, inclusion of sterilizers into the composition, irradiation or heat treatment. Sterile solid compositions can also be prepared which can be solved in sterile water or any other injectable medium immediately before use.

Preferably, pharmaceutical compositions are packaged in unit doses. In such drug form that the preparation is divided into unit doses, each containing a specific quantity of active ingredient. The unit dose form can be a packaged preparation where the packaging contains discrete quantities of the preparation, such as packaged tablets, capsules and powders in vials or ampoules. The unit dose form can also include capsules, tablets, cachets, rhomboid drugs or a certain number thereof included in packaging.

The amount of the active ingredient can change or can be adjusted between 1 and 1000 mg, preferably between 10 and 100 mg in unit dose preparations in accordance with use and the potential of the active ingredient. Pharmaceutical compositions can also contain other compatible therapeutic agents, if necessary.

The effective dose of the composition applied according to the present invention and the rate of dosage to prevent, suppress or hinder arthritis depend on a number of factors. Suitable doses should be obligatorily determined by professionals. In general, it is the attendant physician who specifies the proper dose depending on the age, body weight and any other individual factors of the person to be treated. Daily dose levels vary between about 0.1 and 1000 mg/kg body weight, preferably about 1 to 500 mg/kg/day and more preferably about 50 to 250 mg/kg/day. For safety reasons, the entire daily dose can be divided and administered in portions during the day, if necessary.

When the pharmaceutical compositions include another active ingredient besides Avemar®, the other agent can be selected from the following group: corticosteroids, anti-inflammatory agents, anti-rheumatic agents, immune suppressors, antimetabolites and immune modulators. The list of the compounds pertaining to these categories can be found in the following manual: “Comprehensive Medical Chemistry”, Pergamon Press, Oxford, 970-986 (1990). This group includes, for example, sulfasalazine and aminosalycilates (anti-inflammatory agents); cyclosporine, FK-506 and rapamicine (immune suppressors); cyclophosphamide and methotrexate (anti-metabolites); dexamethazone, methylprednisolone, triamcinolone, prednisolone (steroids); and interferons (immune modulators). When Avemar® is applied in combination with one or more further agents, these can be packaged together or they can be administered in combination. The administration of one or more agents in combination with Avemar® is substantially performed simultaneously or subsequently. Professionals can determine the most suitable method of administration depending on the agents released, the results desired, the patient and the condition to be cured.

Having hereinabove disclosed embodiments of the present invention, those skilled in the art will recognize that this disclosure is only exemplary such that various alternatives, adaptations and modifications are within the scope of the invention, and are contemplated by the Applicants. Accordingly, the present invention is not limited to the specific embodiments as illustrated above, but is defined by the following claims.

1. Use of a fermented wheat germ extract (Avemar®) for the manufacture of a medicament for treating or preventing or alleviating arthritis.

2. The use according to claim 1 wherein arthritis is rheumatoid arthritis.

3. Use of a fermented wheat germ extract (Avemar®) and an anti-inflammatory agent for the manufacture of a medicament for treating or preventing or alleviating arthritis.

4. The use according to claim 3 wherein the anti-inflammatory agent is a non-steroidal anti-inflammatory agent.

5. The use according to claim 5 wherein the non-steroidal anti-inflammatory agent is diclophenac.

6. A pharmaceutical composition comprising an effective amount of fermented wheat germ extract (Avemar®) in combination with an anti-inflammatory agent and a pharmaceutically acceptable carrier.

7. The pharmaceutical composition according to claim 6 wherein the anti-inflammatory agent is a non-steroidal anti-inflammatory agent.

8. The pharmaceutical composition according to claim 7 wherein the non-steroidal anti-inflammatory agent is diclophenac.

9. A method of treating or preventing or alleviating arthritis in a mammal including human comprising administering to said mammal, in which such treatment or prevention or alleviation is desired, an effectice amount of fermented wheat germ extract (Avemar®).

10. The method of claim 9 comprising further administering an anti-inflammatory agent.
